(19) United States
(12) Patent Application Publication
(10) Pub. No.: US 2008/0021118 A1
(43) Pub. Date: Jan. 24, 2008

(54) HYDROCARBON RECOVERY IN THE FISCHER-TROPSCH PROCESS

(75) Inventors: Juan Inga, Sapulpa, OK (US); Tsungani Record, Tulsa, OK (US)

Correspondence Address: BAKER & MCKENZIE LLP, Pennzoil Place, South Tower, 711 Louisiana, Suite 3400, HOUSTON, TX, 77002-2716, US

(73) Assignee: SYNTROLEUM CORPORATION, Tulsa, OK (US)

(21) Appl. No.: 11/781,358

(22) Filed: Jul. 23, 2007

Related U.S. Application Data
(60) Provisional application No. 60/820,028, filed on Jul. 21, 2006.

(51) Int. Cl. C07C 27/00

(52) U.S. Cl. 518/700

(57) ABSTRACT

The invention provides an improved hydrocarbon recovery process of the Fischer-Tropsch process overhead stream using a scrubber system. Described is a process for recovering Fischer-Tropsch hydrocarbons from a reactor exit gas produced from a Fischer-Tropsch synthesis operation. The process includes (a) passing the reactor exit gas to a gas/liquid contactor; and (b) withdrawing a lean tail gas stream, a light hydrocarbon stream, and a water stream from the scrubber.

### PRIOR RELATED APPLICATIONS

This Application claims priority over U.S. Provisional Application No. 60/820,028, filed Jul. 21, 2006, which is incorporated herein in its entirety.

### FEDERALLY SPONSORED RESEARCH STATEMENT

Not applicable.

### REFERENCE TO MICROFICHE APPENDIX

Not applicable.

### FIELD OF THE INVENTION

The invention relates to an improved process for recovering hydrocarbons from a Fischer-Tropsch overhead stream using a scrubber system.

### BACKGROUND OF THE INVENTION

Fischer-Tropsch synthesis operations produce hydrocarbons having a wide range of molecular weights. In general, Fischer-Tropsch hydrocarbons are recovered from the Fischer-Tropsch reactor along with a tail gas. Tail gas represents an overhead product from the Fischer-Tropsch synthesis operation containing the light hydrocarbon fraction plus carbon oxides, hydrogen, and water vapor. Tail gas is generally considered a low value product which may be recycled to the reactor or burned locally as fuel. Although much of the hydrocarbons present in the tail gas are low value hydrocarbons, such as methane, ethane, and propane, the tail gas may also contain significant amounts of butane and propane, as well as some hexane, pentane, and octane. These hydrocarbons represent commercially valuable products which are desirable to recover from the tail gas. Unfortunately, there are few economically attractive methods for recovering these heavy ends from the tail gas.

Two conventional approaches are available for recovering the heavy ends from the tail gas. Cryogenic cooling may be used to condense and remove the heavy ends from Fischer-Tropsch tail gas. A second approach is to use a circulating lean oil in an adsorption tower to adsorb the hydrocarbons in the tail gas to form rich oil. However, conventional lean oil recovery methods require a regeneration loop where the valuable hydrocarbon products are extracted from the rich oil prior to re-circulating the regenerated lean oil back to the adsorption tower.

An economical and improved recovery process for recovering the hydrocarbons from the tail gas of a Fischer-Tropsch reactor would be beneficial.

### SUMMARY OF THE INVENTION

Embodiments of the invention provide a separation system which combines heat and mass transfer in a single piece of equipment to recover light hydrocarbons as well high boiling point components.

In another embodiment, a process for recovering Fischer-Tropsch hydrocarbons from a reactor exit gas produced from a Fischer-Tropsch synthesis operation is described. The method includes: (a) passing the reactor exit gas to a gas/liquid contactor; and (b) withdrawing a lean tail gas stream, a light hydrocarbon stream, and a water stream from the scrubber. The method may further include recycling at least a portion of the water stream back to the gas/liquid contactor. In some embodiments, the reactor exit gas stream comprises methane and heavier hydrocarbons up to C₁₈, carbon oxides, hydrogen, nitrogen and water vapor and the light hydrocarbon stream comprises C₁ to C₄₀ hydrocarbons. In a preferred embodiment, the percent recovery of light hydrocarbons from the FTR reactor exit gas is >90%. In an alternate embodiment, the gas/liquid contactor is operated at a temperature and pressure so that the light hydrocarbon stream contains at least 90% of the hydrocarbons present in the reactor exit gas. In another alternate embodiment, the operating temperature of the gas/liquid contactor is above 2° C. and the operating pressure of the scrubber is lower than the pressure of the FTR. The recycled water may be cooled prior to entering the gas/liquid contactor to a temperature of above 2° C. The gas/liquid contactor may be packed with random or structured packing. In a preferred embodiment, the gas/liquid contactor is a scrubber.

### BRIEF DESCRIPTION OF THE DRAWINGS

FIG. 1 is a flow diagram for a standard hydrocarbon recovery method.

FIG. 2 is a flow diagram of an embodiment of the inventive hydrocarbon recovery method.

FIG. 3 is a flow diagram of an alternate embodiment of the inventive hydrocarbon recovery method.

### DESCRIPTION OF EMBODIMENTS OF THE INVENTION

Unless otherwise specified, all quantities, percentages and ratios herein are by weight.

Three basic techniques may be employed for producing a synthesis gas, or syngas, which is used as the starting material of a Fischer-Tropsch (“FT”) reaction. These include oxidation, reforming and autothermal reforming. As an example, a Fischer-Tropsch conversion system for converting hydrocarbon gases to liquid or solid hydrocarbon products using autothermal reforming includes a synthesis gas unit, which includes a synthesis gas reactor in the form of an autothermal reforming reactor (“ATR”) containing one or more reforming catalysts, such as a nickel-containing catalyst. A stream of light hydrocarbons to be converted, which may include natural gas, is introduced into an ATR along with an oxygen-containing gas which may be compressed air, other compressed oxygen-containing gas, or pure oxygen. The ATR reaction may be adiabatic, with no heat being added or removed from the reactor other than from the feeds and the heat of reaction. The reaction is carried out under sub-stoichiometric conditions whereby the oxygen/steam/gas mixture is converted to syngas.

Known autothermal processes for the production of synthesis gas are disclosed in, for example, U.S. Pat. Nos. 6,085,512; 6,155,039; and 4,833,170, the disclosures of each of which are incorporated herein by reference.

The Fischer-Tropsch reaction for converting syngas, which is composed primarily of carbon monoxide (CO) and hydrogen gas (H₂), may be characterized by the following general reaction:

2nH₂+nCO→(—CH₂—)ₙ+nH₂O (1)

Non-reactive components, such as nitrogen, may also be included or mixed with the syngas. This may occur in those instances where air, enriched air, or some other non-pure oxygen source is used during the syngas formation.

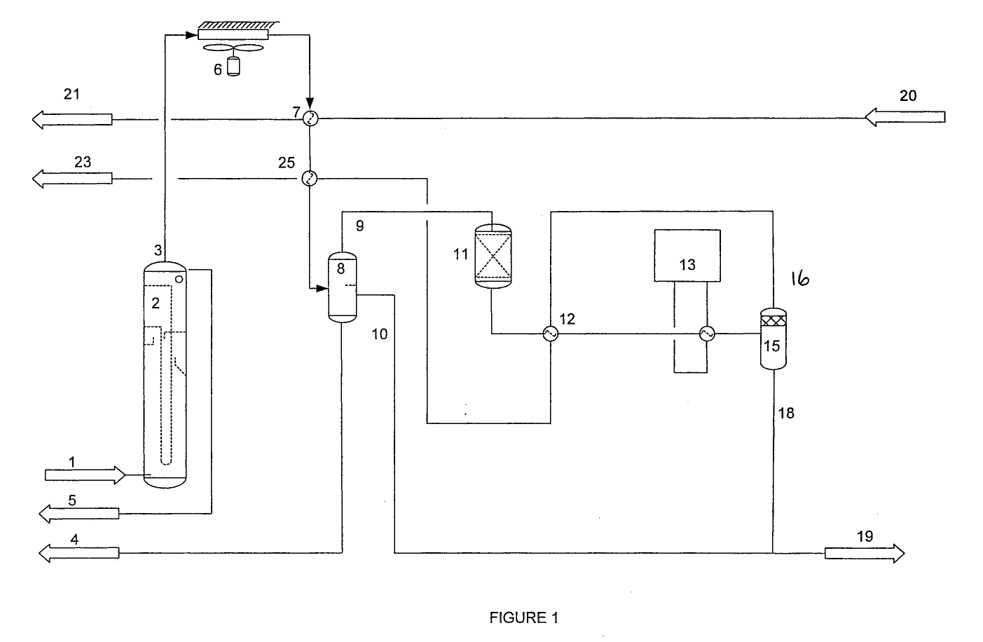

Referring to FIG. 1, in a typical embodiment, syngas (1) from a syngas generator is delivered to a Fischer-Tropsch reactor (2) (FTR) containing a Fischer-Tropsch catalyst to produce a mixture of hydrocarbons. The reactor exit gas leaves the reactor via the overhead (3). The liquid products leave the reactor via the bottoms (5). Numerous Fischer-Tropsch catalysts may be used in carrying out the reaction. These include cobalt, iron, ruthenium as well as other Group VIIIB transition metals or combinations of such metals, to prepare both saturated and unsaturated hydrocarbons. The Fischer-Tropsch catalyst may include a support, such as a metal-oxide support, including silica, alumina, silica-alumina or titanium oxides. For example, a Co catalyst on transition alumina with a surface area of approximately 100-200 m²/g may be used in the form of spheres of 20-150 μm in diameter. The Co concentration on the support may also be between about 15 to about 30%. Certain catalyst promoters and stabilizers may be used. The stabilizers include Group IIA or Group IIIB metals, while the promoters may include elements from Group VIII or Group VIIB. The Fischer-Tropsch catalyst and reaction conditions may be selected to be optimal for desired reaction products, such as for hydrocarbons of certain chain lengths or number of carbon atoms. Any of the following reactor configurations may be employed for Fischer-Tropsch synthesis: fixed bed, slurry bed reactor, ebullating bed, fluidizing bed, or continuously stirred tank reactor (CSTR). The FTR may be operated at a pressure of 100 to 550 psia and a temperature of 190.5° C. to 371° C. The reactor gas hourly space velocity (“GHSV”) may be from 1000 to 8000 hr⁻¹. Syngas useful in producing a Fischer-Tropsch product useful in the invention may contain gaseous hydrocarbons, hydrogen, carbon monoxide and nitrogen with H₂/CO ratios from about 1.8 to about 2.4. The hydrocarbon products derived from the Fischer-Tropsch reaction may range from methane (CH₄) to high molecular weight paraffinic waxes containing more than 100 carbon atoms.

Examples of Fischer-Tropsch systems are described in U.S. Pat. Nos. 4,973,453; 5,733,941; 5,861,441; 6,130,259, 6,169,120 and 6,172,124, the disclosures of which are herein incorporated by reference.

When the FTR is operated below about 260° C., the liquid products (5) from the Fischer-Tropsch reaction include hydrocarbons ranging from methane (CH₄) to high molecular weight paraffinic waxes containing more than 100 carbon atoms.

The reactor exit gas (3) may comprise nitrogen, carbon dioxide, carbon monoxide, hydrogen, water and light hydrocarbons typically having a molar composition range of about 15 to 90% N₂, 5 to 10% CO₂, 0.5 to 15% CO, 1 to 30% H₂, 0.1 to 10% H₂O and the remainder hydrocarbons. Thus, the reactor exit gas contains inert non-combustible components in a range of about 20 to 94 mole % with the remainder being water and combustible components. As such, the reactor exit gas has a heating value in a range of about 2,500 to 15,800 kJ/m³. Inert non-combustible components are defined herein as components which will not react exothermically with oxygen. Such components include nitrogen, argon, carbon dioxide and water. Combustible components are defined herein as components which may react exothermically with oxygen at elevated temperatures. Such components include carbon monoxide, hydrogen, alcohols, methane and heavier hydrocarbons.

The reactor exit gas (3) is cooled using a cooler. In some embodiments, the cooler is an air cooler (6), water coolers (7) and feed-product exchanger (25), or a combination thereof. Upon exiting the cooler (25), the tail gas is at a temperature of about from 25° C. to 40° C. The cooled tail gas is fed to a three phase separator (8). The three phase separator (8) is configured to permit the separation of a gas phase and two liquid phases within a bottom portion of the separator (8). An FT produced water stream (4) exits the bottom, a light hydrocarbon stream (10) is withdrawn from a side port and a tail gas (9) exits the top. The tail gas (9) is fed to a dehydrator (11) to remove entrained water and then further cooled by a cooler (12) and a refrigeration system (13). In a preferred embodiment, the dehydrator (11) contains alumina fill to extract any remaining water. The refrigeration system (13) is preferably a propane refrigeration system which cools the tail gas to a temperature of about −33° C., condensing the majority of the remaining hydrocarbons in the gas. After exiting the refrigeration system (13), the tail gas is fed to a final separator (15) which operates to separate a gas phase and a liquid phase. A lean tail gas (16) exits the top of the final separator (15) and exchanges heat with the dehydrated tail gas in cooler (12) and the reactor exit gas in cooler (25) and the lean tail gas (23) is heated to a temperature of about 30° C. to 45° C. The warm lean tail gas (23) may then be used as a fuel source to generate power or may then be further processed. The typical molar (mol %) composition of the cooled lean tail gas at about 19 atms and 38° C. is about 84.3% N₂, 4.5% CO₂, 2.0% CO, 4.3% H₂, 3.1% CH₄ and 0.8% C₂₊.

A light hydrocarbon stream (18) exits the bottom of the separator (15) and is combined with the light hydrocarbon stream (10) from the three-phase separator (8). The combined light hydrocarbon stream (19) may be sent for further processing such as stabilization, hydrogenation, etc. Recovery using this approach can recover approximately between 85% to 88% of the hydrocarbons from the reactor exit gas (3) in the light hydrocarbon stream (19). The light hydrocarbon stream (18) includes hydrocarbons as light as propane. The typical temperature is −20° C. The typical pressure is 370 psig. The coolant (20) of water cooler (7) may be cooling water or any process stream

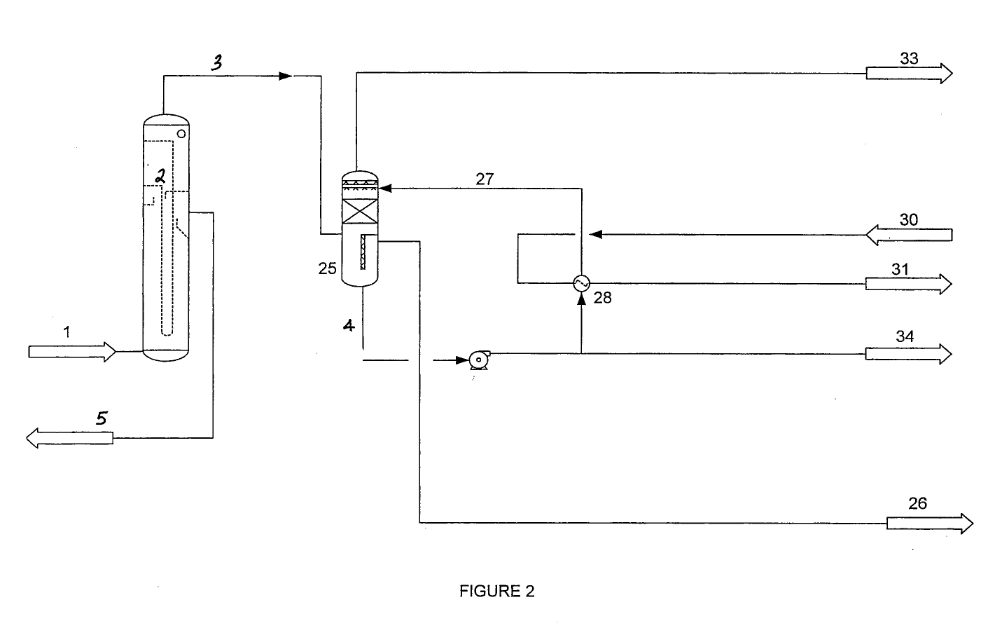

Referring to FIG. 2, a preferred embodiment of the invention has syngas (1) being fed to the FTR (2). Parts or features which are the same as or similar to those in FIG. 1, are indicated with the same reference numerals. The reactor exit gas (3) leaves the reactor via the overhead. The liquid products (5) exit the bottom of the FTR (2). The reactor exit gas (3) is fed to a gas/liquid contactor (25). In a preferred embodiment, the gas/liquid contactor (25) is a scrubber. The scrubber is configured to permit the separation of a gas phase and two liquid phases within a bottom portion of the scrubber (25). The scrubber may be packed with packing supplied by Koch-Glitsch or Jaeger. The packing may be random, structured or a combination. An FT water stream (34) exits the bottom, a light hydrocarbon stream (26) is withdrawn from a side port and a lean tail gas (33) exits the top. In some embodiments, the FT water (4) is divided into two streams (27) and (34). FT water stream (27) is cooled by heat exchanger (28) and returned to the scrubber (25). FT water stream (34) is sent for further processing that could include recovery of oxygenates and further treatment prior to disposal or may be used as a cooling medium within the FT plant. The coolant (30) of heat exchanger (28) may be cooling water or a FT produced stream, including, for example, a hydrocarbon stream. Recovery of the hydrocarbons from the reactor exit gas (3) is approximately greater than 88%, preferably greater than 89%, more preferably greater than 90% in the light hydrocarbon stream (26). This embodiment requires less equipment and less power consumption than the process illustrated in FIG. 1.

The following table lists the equipment for the processes shown in FIGS. 1 and 2 described above and compares the performance requirements and capital investment.

TABLE 1
Equipment Size for a 45,000 bpd GTL Plant

| | FIG. 1 | | | | | |
|---|---|---|---|---|---|---|
| | (Comparative | | | | | |
| Equipment | Example) | Design size | | FIG. 2 | Design size | |
| Air cooler | 6 | 398 | MM Btu/h | not | | |
| | | | | needed | | |
| Tail Gas water | 7 | 47.5 | MM Btu/h | 28 | 86.4 | MM Btu/h |
| cooler | | | | | | |
| Tail gas exchanger | 22 | 52.6 | MM Btu/h | not | | |
| | | | | needed | | |
| 3-phase separator | 8 | 140 | m³ | 25 | 235 | m³ |
| | | | | (Scrubber) | | |
| Tail gas dryers | 11 | | | not | | |
| | | | | needed | | |
| Dry tail gas | 12 | 74 | MM Btu/h | not | | |
| exchanger | | | | needed | | |
| Propane | 13 | 7 | MW motor | not | | |
| refrigeration | | | | needed | | |
| system | | | | | | |
| Propane cooler | 14 | 52.8 | MM Btu/h | not | | |
| | | | | needed | | |
| Final separator | 15 | 63 | m³ | not | | |
| | | | | needed | | |

| | | | |
|---|---|---|---|
| Water pump | not needed | 29 | 27.68 hp; 1780 |
| | | | gpm |

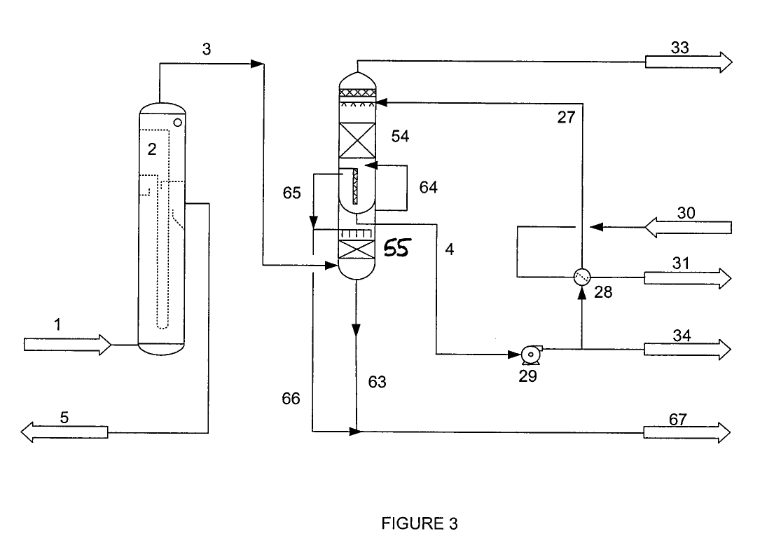

Referring to FIG. 3, in another alternate embodiment of the invention syngas (1) is fed to the FTR (2). Parts or features which are the same as or similar to those in FIGS. 1 and 2, are indicated with the same reference numerals. The reactor exit gas (3) leaves the reactor via the overhead. The liquid products (5) exit the bottom of the FTR (2). The reactor exit gas (3) may contain heavy hydrocarbons. The presence of these heavy hydrocarbon species in the reactor exit gas (3) may be increased by the use of a FT catalyst that has a high alpha value (above 0.9), the operation of the FT reactor at lower temperatures (200-215° C.), the operation of the FT reactor at low pressures (below 400 psig) or under non-operating conditions such as startup or catalyst treatment. It is necessary to remove these heavy hydrocarbon species before cooling to avoid precipitation of the waxy material. The reactor exit gas (3) enters a preliminary gas/liquid contactor (55) that contains packing material. This preliminary gas/liquid contactor (55) may be part of the main gas/liquid contactor (54), as shown, or could be a separate vessel. In a preferred embodiment, both the preliminary gas/liquid contactor (55) and main gas/liquid contactor (54) are scrubbers. The liquid in the preliminary scrubber (55) is a light oil (65) that is condensed and collected in main scrubber (54). The temperature of the reactor exit gas (3) is lowered by the light oil (65). A portion of the light oil is vaporized, condensing the heavy hydrocarbons waxy material of the reactor exit gas (3). A mixture (63) of the heavy hydrocarbons waxy material is mixed with the remaining liquid light oil and exits the lower part of the preliminary gas/liquid contactor (55). A mixture (64) of the non-condensed vapors of the reactor exit gas (3) and the vaporized light oil are sent to the upper part of the main scrubber (54). The main scrubber (54) is configured to permit the separation of a gas phase and two liquid phases within a bottom portion of the main scrubber (54). The preliminary scrubber (55) is configured to permit the separation of a gas phase and a liquid phase within a bottom portion of the preliminary scrubber (55). The scrubbers (54) and (55) may be packed with packing supplied by Koch-Glitsch or Jaeger. The packing may be random, structured or a combination. An FT water stream (4) exits the bottom of the main scrubber (54), a lean tail gas (33) exits at the top of the main scrubber (54) and a light hydrocarbon stream (65) is withdrawn from a side port of the main scrubber (54). The light oil (65) is split into a first fraction going to the bottom part of the preliminary scrubber (55) to cool the reactor exit gas (3) and condense the waxy material while a second fraction (66) is sent for further processing. Ultimately, the mixture (63) of the heavy hydrocarbons waxy material and the liquid light oil from the preliminary scrubber (55) will be combined with the second fraction (66) to be a FT product (67) sent for further processing, such as but not limited to hydrotreatment or hydrocracking. In some embodiments, the FT water (4) is divided into a first fraction (27) and a second fraction (34). The first fraction FT water (27) is cooled by heat exchanger (28) and returned to the main scrubber (54). The second fraction FT water stream (34) is sent for further processing, such as, but not limited to recovery of oxygenates, treatment prior to disposal or usage as a cooling medium within the FT plant. The coolant (30) for the heat exchangers (28) may be cooling water or a FT produced stream, including, for example, a hydrocarbon stream. Recovery of the hydrocarbons from the reactor exit gas (3) is approximately greater than 88%, preferably greater than 89%, more preferably greater than 90% in the light hydrocarbon stream (67). The embodiment shown in FIG. 3 requires less equipment and less power consumption than the process illustrated in FIG. 1.

While the invention has been described with respect to a limited number of embodiments, the specific features of one embodiment should not be attributed to other embodiments of the invention. No single embodiment is representative of all aspects of the inventions. Moreover, variations and modifications therefrom exist. For example, other stripping mediums may be used to increase hydrocarbon recovery in the scrubber. Additionally, heat exchangers and preheaters may be designed for maximum heat efficiency. The appended claims intend to cover all such variations and modifications as falling within the scope of the invention.

What is claimed is:

1. A process for recovering Fischer-Tropsch hydrocarbons from a reactor exit gas produced: from a Fischer-Tropsch synthesis operation which comprises:
(a) passing the reactor exit gas to a gas/liquid contactor;
(b) withdrawing a lean tail gas stream, a light hydrocarbon stream, and a water stream from the scrubber.

2. The process of claim 1 further comprising, recycling at least a portion of the water stream back to the gas/liquid contactor.

3. The process of claim 1 further comprising, recycling at least a portion of the light hydrocarbon stream back to the gas/liquid contactor.

4. The process of claim 1 further comprising, recycling at least a portion of the water stream and at least a portion of the light hydrocarbon stream back to the gas/liquid contactor.

5. The process of claim 1, wherein the reactor exit gas stream comprises methane and heavier hydrocarbons up to C₁₈, carbon oxides, hydrogen, nitrogen and water vapor.

6. The process of claim 1, wherein the light hydrocarbon stream comprises C₁ to C₄₀ hydrocarbons.

7. The process of claim 1, wherein the percent recovery of light hydrocarbons from the FTR reactor exit gas is ≧90%.

8. The process of claim 1, wherein the gas/liquid contactor is operated at a temperature and pressure so that the light hydrocarbon stream contains at least 90% of the hydrocarbons present in the reactor exit gas.

9. The process of claim 1, wherein the operating temperature of the gas/liquid contactor is above 2° C. and the operating pressure of the scrubber is lower than the pressure of the FTR.

10. The process of claim 2, wherein the recycled water is cooled prior to entering the gas/liquid contactor.

11. The process of claim 10, wherein the water is cooled to a temperature of above 2° C.

12. The process of claim 1, wherein the gas/liquid contactor is packed with random or structured packing.

13. The process of claim 1, wherein the Fischer-Tropsch synthesis reaction is carried out in a slurry-type reactor.

14. The process of claim 1, wherein the light hydrocarbon stream is sent to an upgrading operation.

15. The process of claim 1, wherein the gas/liquid contactor is a scrubber.
